(19) United States
(12) Patent Application Publication
(10) Pub. No.: US 2020/0276383 A1
(43) Pub. Date: Sep. 3, 2020

(54) TUBE MANAGEMENT DEVICE FOR AN INFUSION SET

(71) Applicant: Eli Lilly and Company, Indianapolis, IN (US)

(72) Inventors: Daniel Morris HARTMANN, Arlington, MA (US); Gavin Miles MCKEOWN, Bedford, MA (US); Sean Matthew PSZENNY, Cambridge, MA (US)

(21) Appl. No.: 16/645,711

(22) PCT Filed: Sep. 10, 2018
(86) PCT No.: PCT/US2018/050188

§ 371 Date: Mar. 9, 2020

Related U.S. Application Data
(60) Provisional application No. 62/557,877, filed on Sep. 13, 2017.
(60) Provisional application No. 62/665,225, filed on May 1, 2018.

(51) Int. Cl. A61M 5/142; A61M 5/14; A61M 39/08

(52) U.S. Cl. CPC A61M 5/14248; A61M 2039/087; A61M 39/08; A61M 5/1418

(57) ABSTRACT

Tube management devices are disclosed for use with an infusion set having a flexible tube. An exemplary device includes a central spindle defining an axis, a base member, and an upper member. The base member and upper member may project radially outwardly from the central spindle and may be axially spaced apart. The device may include a plurality of retainer members disposed on at least one of the base member and the upper member. The central spindle, the base member, the upper member, and the plurality of retainer members define a toroidal space sized to receive a variable length of the flexible tube.

### CROSS REFERENCE TO RELATED APPLICATIONS

This application claims priority to Provisional Application No. 62/557,877, filed Sep. 13, 2017, and Provisional Application No. 62/665,225, filed May 1, 2018, each entitled TUBE MANAGEMENT DEVICE FOR AN INFUSION SET, both of which are hereby incorporated by reference in their entirety.

### FIELD OF THE DISCLOSURE

The present disclosure relates to infusion sets for a delivering a medication to a patient, and more particularly to a tube management device for an infusion set.

### BACKGROUND

The present disclosure relates to infusion sets for a delivering a medication to a patient. Infusion sets typically include tubing that leads from a medication delivery device, such as a pump, to a cannula that is inserted into the subcutaneous tissue of the patient. Such infusion sets are often used to inject insulin or other suitable medications.

A typical infusion set also includes a connector hub disposed between the medication pump and the cannula. Connector hubs include two mating parts that allow the pump to be connected and disconnected from the cannula without having to remove the cannula from the subcutaneous tissue. A conventional connector hub includes a female part having a septum and a male part having a needle that pierces the septum when the male and female parts are joined together.

In some infusion sets, the connector hub also functions as an insertion hub with the cannula being located at the same hub as the septum/needle assembly connection. In other infusion sets, the connection hub is separate from the insertion hub. In an infusion set having separate connection and insertion hubs, a length of tubing will extend from the connection hub housing the septum/needle assembly to the insertion hub housing the cannula. In both types of infusion sets, a length of tubing will extend from the pump to the connection hub.

In both types of infusion sets, the hubs are typically designed to attach to the skin of the user with an adhesive. Adhesively securing the hub associated with the cannula to the skin of the user helps reduce the movement of the cannula relative to the tissue of the patient and thereby helps prevent inadvertent withdrawal of the cannula. When a separate insertion hub and connection hub are used, the adhesive attachment of the connection hub to the patient can provide strain relief with regard to the tube leading to the insertion hub. Although such infusion set arrangements can be effective, they also have several shortcomings.

One drawback when using a system having only a pump and a combined connection and insertion hub is that there is no second hub disposed between the pump and combined connection/insertion hub to provide strain relief on the tubing leading to the hub housing the cannula.

For infusion sets having separate connection and insertion hubs, the length of tubing connecting the connection hub to the insertion hub is typically fixed in length. This fixed length of tubing may not be optimal for all patients. For example, it may be desirable to use a shorter length of tubing for small children than for an adult for optimal comfort when placing the hubs on the patient.

Another drawback is that once the connection hub has been affixed to the body, it cannot be repositioned. Thus, the location and angle at which the tube leaves the hub and the length of tubing between the connection hub and the insertion hub are also fixed. The angle at which the tubing protrudes from the connection hub toward the pump may limit the ability of the user to wear the pump in different locations on their body. The fixed length of tubing between the connection hub and insertion hub may also present difficulties. For example, a relatively long length of tubing may be uncomfortable or become snagged on clothing while a relatively short length of tubing may pull on the insertion site and cause discomfort.

A drawback to using separate connection and insertion hubs is that medication remains in the fixed length of tubing between the two hubs when the pump is detached. The medication in this fixed length of tubing may spend an extended time in this length of tubing where it may be subjected to elevated temperatures such as when the patient takes a bath. Additionally, if there is any back-flow from the patient into the infusion set, the medication within this length of tubing may be contaminated and may degrade.

Another drawback is that conventional connection hubs and insertion hubs provide the user with limited choice. For example, infusion sets are typically provided with a fixed length of tubing and the user is limited to a selection of a short tube length (typically between 16 inches (40.6 cm) and 18 inches (45.7 cm)), a medium tube length (typically between 23 inches (58.4 cm) and 24 inches (61.0 cm)), long tube length (typically between 31 inches (78.7 cm) and 32 inches (81.3 cm)) and extra long tube length (typically between 42 inches (106.7 cm) and 43 inches (109.2 cm)). Longer lengths are useful for larger individuals and for those patients who want to wear a pump at a convenient location, such as on a waist belt, and infuse at a remote location, such as a forearm. Users are encouraged to rotate infusion sites to prevent scar tissue formation. Thus, a patient may want to use a long length of tubing for one infusion site and a shorter length for the next infusion site.

Infusion sets are typically purchased in boxes having multiple sets (e.g., ten infusion sets per box), and multiple, identical boxes are often purchased in a single order to maximize insurance benefits. As a result, a patient is often locked into a particular length of tubing and would find it cost-prohibitive to purchase multiple infusion sets having different length tubing.

A way to minimize such drawbacks and enhance patient convenience is desirable.

### SUMMARY

The present disclosure provides a tube management device that allows a user to easily and conveniently manage a length of flexible tubing used to transfer medication.

According to an embodiment of the present disclosure, a tube management device is disclosed for an infusion set having a flexible tube for conveying medication between a reservoir and an infusion site on a patient. The device includes a central spindle defining an axis, a base member projecting radially outwardly from the central spindle, an upper member projecting radially outwardly from the central spindle, and a plurality of retainer members disposed on at least one of the base member and the upper member. The base member and the upper member are axially spaced apart. The central spindle, the base member, the upper member, and the plurality of retainer members define a toroidal space sized to receive a variable length of the flexible tube. The plurality of retainer members restrict radially outward movement of a length of the flexible tube wound about the central spindle.

According to another embodiment of the present disclosure, a tube management device is disclosed for an infusion set having a flexible tube for conveying medication between a reservoir and an infusion site on a patient. The device includes a support member adapted to be secured to the patient and a reel member rotatably mountable on the support member and adapted to receive the flexible tube. When the reel member is mounted on the support member, a rotation of the reel member in a first direction is operable to wind the flexible tube onto the device and a rotation in a second direction is operable to extend the flexible tube from the device. The reel member is detachable from the support member with a length of flexible tube wound on the reel member.

### BRIEF DESCRIPTION OF THE DRAWINGS

The above mentioned and other features of the present disclosure, and the manner of attaining them, will become more apparent to those skilled in the art upon consideration of the following detailed description of embodiments taken in conjunction with the accompanying drawings, wherein:

FIG. 1 is a schematic view of an infusion set with a tube management device according to an illustrative embodiment.

FIG. 2 is a schematic view of another infusion set with a pair of tube management devices according to another embodiment.

FIG. 3 is a perspective view of an exemplary embodiment of a tube management device.

FIG. 4 is a partially exploded view of the tube management device of FIG. 3.

FIG. 5 is a perspective view of a device similar to that of FIG. 3 further including a mounting member releasably securable to the tube management device.

FIG. 6 is a top view of a mounting member of FIG. 5.

FIG. 7 is a partial detail view of the mounting member of FIG. 6.

FIG. 8 is a bottom view of a device releasably securable to the mounting member of FIG. 6.

FIG. 9 is a partial detail view of the device of FIG. 8.

FIG. 10 is an exploded perspective view of another exemplary embodiment of a tube management device.

FIG. 11 is a perspective view of the device of FIG. 10 with a tube engaged with the device.

FIG. 12 is a perspective view of the device of FIG. 11 after winding additional tubing on the device.

FIG. 13 is a perspective view of the device of FIG. 12 with an attached cap, wherein the reel member is disengaged from the support member with tubing secured on the reel member.

FIG. 14 is an exploded perspective view of another exemplary embodiment of a tube management device.

FIG. 15 is a cross-sectional view of the central spindle of the device of FIG. 14 taken along line A of FIG. 14.

FIG. 16 is a cross-sectional view of the base member of the device of FIG. 14 taken along line B of FIG. 14.

FIG. 17 is a top perspective view of the assembled spindle and base member of the device of FIG. 14.

FIG. 18 is a bottom perspective view of the upper member of the device of FIG. 14.

FIG. 19 is an exploded perspective view of another exemplary embodiment of a tube management device.

FIG. 20 is a bottom perspective view of the upper member of the device of FIG. 19.

FIG. 21 is a perspective view of another exemplary embodiment of a tube management device.

FIG. 22 is an exploded perspective view of the device of FIG. 21.

FIG. 23 is a partially exploded, bottom perspective view of the device of FIG. 21.

Corresponding reference characters indicate corresponding parts throughout the several views. Although the exemplifications set out herein illustrate embodiments of the invention, in several forms, the embodiments disclosed herein are not intended to be exhaustive or to be construed as limiting the scope of the invention to the precise forms disclosed.

### DETAILED DESCRIPTION

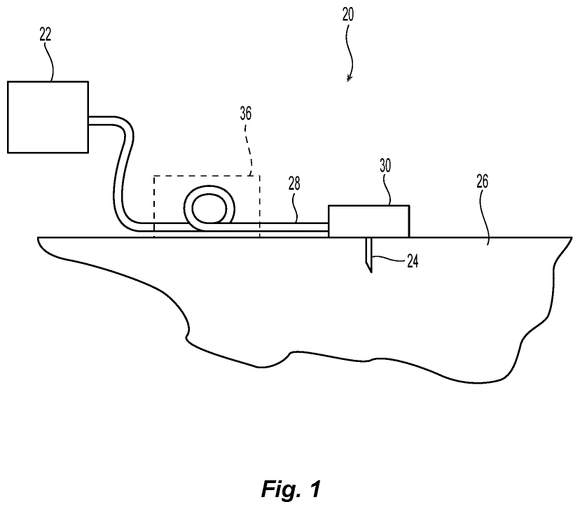

FIG. 1 schematically depicts an infusion set 20 having a cannula 24 to deliver medication from a medication reservoir 22 to a patient 26. Medication reservoir 22 may take the form of a pump operably coupled with a reservoir whereby the pump discharges the medication, such as insulin for example, which flows through flexible tubing 28 to cannula 24 where it is subcutaneously injected into patient 26. Infusion set 20 has a combined connection/insertion hub 30 which is attached to the skin of the patient. A tube management device 36 is attachable to patient 26 and engages flexible tubing 28 at a location between reservoir 22 and hub 30 as schematically depicted in FIG. 1. In one embodiment, flexible tubing 28 is made of a plastic material.

Several embodiments of a tube management device 36 are disclosed herein and discussed in greater detail below. The use of such a tube management device 36 in the fluid path between reservoir 22 and hub 30 allows the user to better control and secure the tubing 28. Further, device 36 is configured to provide strain relief such that if a force is exerted on tube 28 between reservoir 22 and device 36, such as by an accidental snagging of tube 28, the force will be resisted by device 36, and tube 28 will not exert a force on hub 30 which might otherwise dislodge or disconnect cannula 24. For infusion sets having a particularly long length of tubing between reservoir 22 and hub 30, more than one device 36 may be used to control the excess length of the tubing.

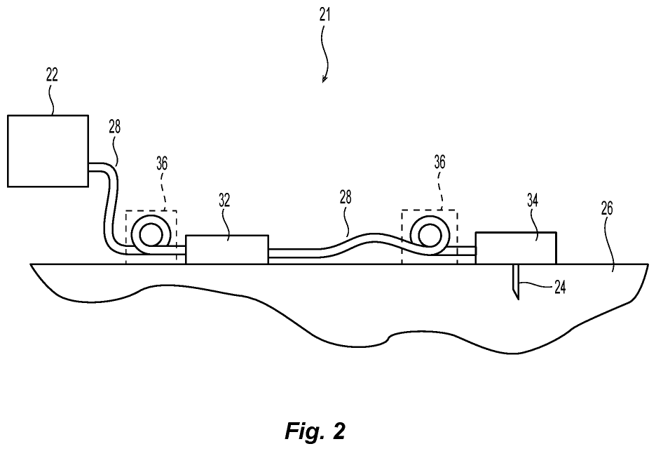

FIG. 2 schematically depicts an infusion set 21 having a connector hub 32 that is physically separate from an insertion hub 34. Connector hub 32 is located in the fluid path between medication reservoir 22 and insertion hub 34 which houses cannula 24. Flexible tubing 28 conveys the medication from reservoir 22 to connection hub 32 and from connection hub 32 to insertion hub 34. Tube management devices 36 may engage tube 28 at locations between reservoir 22 and connection hub 32 and between connection hub 32 and insertion hub 34 as schematically depicted in FIG. 2.

It is noted that infusion sets having separate connection and insertion hubs typically have a relatively short length of tubing between the connection hub 32 and insertion hub 34 that may not require a tube management device. For example, such tube lengths may be between 2 inches (5.1 cm) and 3 inches (7.6 cm). Nevertheless, the use of one or more tube management devices as described herein may be used with the tube at any point in the system where a length of tubing would benefit from the inclusion of such a device.

In an infusion set such as depicted in FIG. 2, tube management devices 36 may be employed both between reservoir 22 and connection hub 32 and between connection hub 32 and insertion hub 34 to meet the needs of the patient.

In various embodiments, both types of infusion sets 20, 21 of FIGS. 1 and 2 have certain advantages. For example, infusion set 21 with its separate connection hub 32 separates the functionality of connecting and disconnecting the pump from the insertion hub and thereby reduces the likelihood that the act of connecting or disconnecting the pump at the connection hub will disturb the cannula. More importantly, it also allows the separate connection hub 32 to act as strain relief for the insertion hub 34. However, the tubing between the insertion hub and connection hub is filled with medication and remains connected to the patient even when the rest of the infusion set is removed. The medication contained within this length of tubing (which may be approximately 15 microliters in some infusion sets) may therefore be exposed to different environmental conditions than the remainder of the medication transported by the set. For example, it may be subjected to higher temperatures when the user takes a bath. Moreover, if there is any back-flow from the patient into the infusion set, the medication contained in this length of tubing between the insertion hub and the connection hub may be contaminated and may degrade.

The use of a tube management device 36 between the reservoir 22 and a combined hub 30 (FIG. 1) can provide strain relief for the cannula while eliminating the length of tubing between the insertion hub 34 and connection hub 32 that retains medication when the remainder of the set is disconnected (FIG. 2).

By providing a tube management device 36 between the reservoir and combined hub 30, the tube management device 36 is configured to prevent strain from being imparted to the inserted cannula 24. Without such a strain relief device in place, strain may be imparted to the cannula 24 when the user pulls on the infusion tubing 28, which may happen if the user adjusts a waist-belt supporting the pump, snags the tubing on an object, drops the pump, or removes clothing supporting the pump. However, if the tube management device 36 is between the reservoir and combined hub 30 it will absorb such strain and resist dislodgement of the cannula from the patient's body.

The use of a tube management device 36 as described herein eliminates or substantially reduces the need for a separate connection hub to provide strain relief and thereby provides strain relief without creating a length of tubing that can lead to the degradation of the medication as described above.

The use of a tube management device 36 also facilitates the use of a relatively long length of tubing between reservoir 22 and either a connection hub 32 or a combined connection/insertion hub 30. The use of a relatively long tube 28 between the connection hub 32 and reservoir 22, or, between the insertion hub 30 and reservoir 22, allows the user greater freedom in choosing the wear location of the reservoir 22 and also greater freedom in selecting the insertion site. The use of such longer tube lengths, however, may present problems when the locations chosen do not require the full length of the tube being used. In such a case, the use of one or more tube management devices 36 is beneficial.

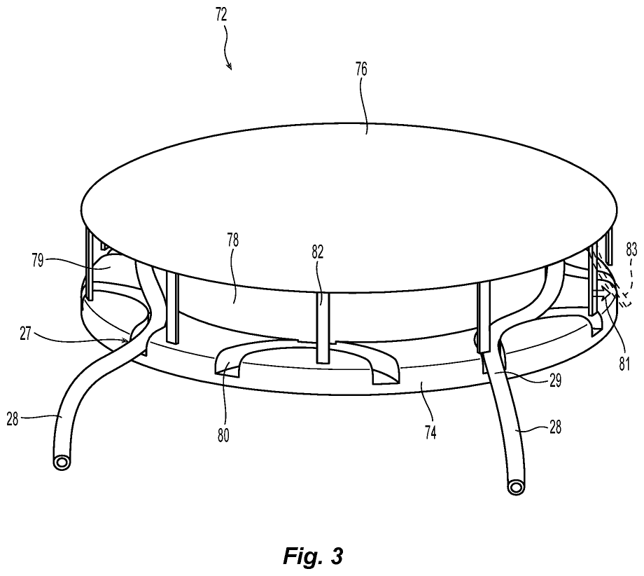

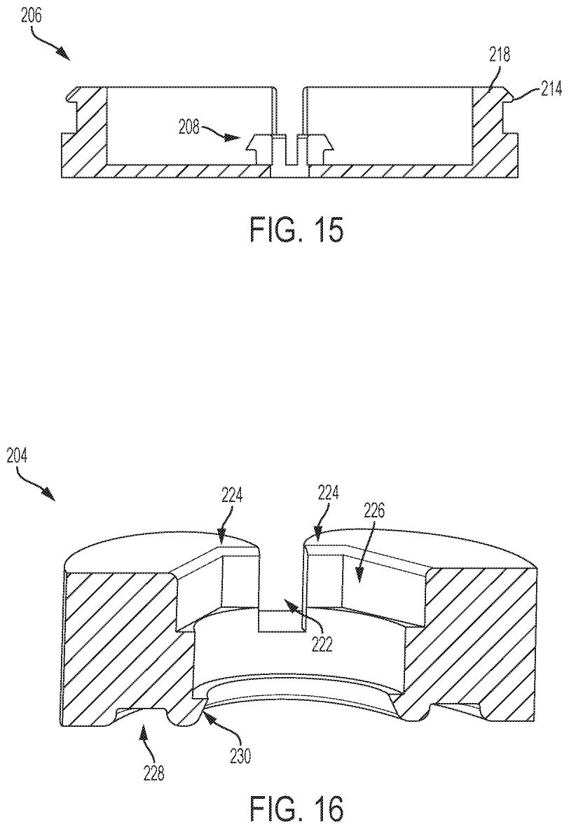

Several embodiments of suitable tube management devices are illustrated in FIGS. 3-23 and are discussed herein. These devices may be engaged with tube 28 and disengaged from tube 28 without disconnecting the terminal end of the tube (i.e., without disconnecting reservoir 22, and without disconnecting from the hub 30, 32 or 34), and without having to thread the terminal end of tube 28 through the device. In other words, a non-terminal portion of the flexible tube 28 may be operably engaged with and disengaged from the devices when the flexible tube 28 is operably connected to convey medication between the reservoir and the infusion site.

Because the disclosed devices are engageable with a tube that is already operably connected to the infusion set without disconnecting either end of the tube, the devices may be positioned at any point in the system where there is excess tubing that would be benefit from the use of a management device or a need for strain relief. Multiple devices may also be used for different sections of tubing in the system, or, with a single section of particularly long tubing.

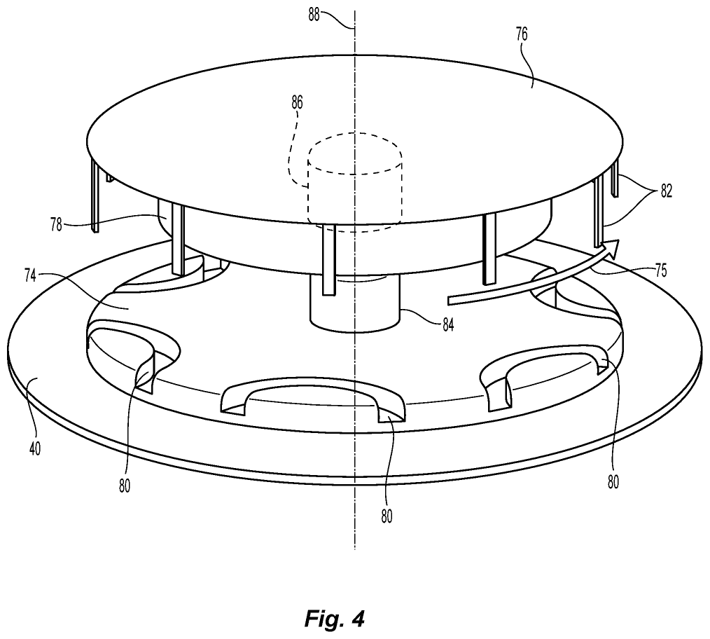

A first embodiment of such a device is depicted in FIGS. 3 and 4 which illustrates a tube management device 72. Tube management device 72 is suited for use in an infusion set having flexible tubing 28 for conveying medication between a reservoir and an infusion site on a patient. Device 72 is shown in an exploded view in FIG. 4 and includes a base member 74 adapted to be attached to the patient, an upper member 76, and a central spool or spindle 78 that is positioned between base member 74 and upper member 76. In the illustrated embodiment, upper member 76 and spindle 78 are integrally formed together and are rotatably mounted on base member 74 by engaging shaft 84 on base member 74 with socket 86 on upper member 76. An adhesive patch 40 is used to secure base member 74 to the patient.

Central spindle 78 defines an axis 88. Both base member 74 and upper member 76 project radially outwardly from spindle 78 with the base member 74 and upper member 76 being axially spaced apart. A plurality of retainer members 82 are disposed on either the base member 74 or the upper member 76 and project toward the other one of the base member and the upper member whereby the central spindle 78, the base member 74, the upper member 76 and the plurality of retainer members 82 define a toroidal space 79. Alternatively, the base member 74 and the upper member 76 may each include retainer members 82 arranged such that the retainer members 82 are spaced apart (similar to FIG. 3) when device 72 is assembled. Toroidal space 79 acts as a holding chamber within which a variable length of the flexible tube 28 may be wound about the central spindle 78 with the plurality of retainer members 82 restricting radially outward movement of the flexible tubing 28 wound about the central spindle 78.

Thus, a user seeking to manage an excess length of tubing 28 may wind the excess length of tubing 28 about spindle 78 within the toroidal volume 79 surrounding spindle 78 and disposed between base member 74 and upper member 76. Base member 74 defines a plurality of grooves 80 circumferentially spaced about the outer perimeter of base member 74. Grooves 80 are illustratively arcuate-shaped channels in base member 74 that grippingly receive tube 28 when tube 28 is positioned in the groove 80. Tube 28 may be engaged with a groove 80 at the location 27 where it enters device 72 and/or the location 29 where it exits device 72 to thereby secure tube 28 to device 72 and to set the length of tubing 28 disposed in device 72. Placing a plurality of grooves 80 about the outer perimeter of base member 74 provides flexibility in the location where tube enters and leaves the device. Grooves 80 may also be disposed along the outer perimeter of upper member 76.

It is noted that, while the user may engage tubing 28 with a groove 80 at both the location where tube 28 enters the device and where tube 28 exits the device, the user may alternatively engage tube 28 at only one location. The wrapping of tube 28 about spindle 78 may, in some situations, also be sufficient by itself to provide strain relief without the use of grooves 80. In an exemplary configuration, a user may employ device 72 to engage tube 28 in a groove 80 at the point nearest the insertion hub and to leave the tube un-engaged with a groove 80 at the point nearest the reservoir 22. In this example, the point of engagement with a groove 80 nearest the insertion hub provides strain relief for the insertion hub and the loose end facing the reservoir allows excess tubing to be more easily enter or be removed from toroidal cavity 79, e.g., by wrapping or unwrapping tube 28 on spindle 78.

The illustrated plurality of circumferentially spaced retainer members 82 extending between upper member 76 and base member 74 along the outer radial edge of upper member 76 to thereby define the outer radial limit of toroidal space 79 are formed out of a material having sufficient resilient flexibility that allows members 82 to be bent outwardly and inwardly to allow tube 28 to be moved into or out of toroidal space 79.

More specifically, the illustrated retainer members 82 are resiliently flexible between an at rest position and a flexed position. The flexible retainer members 82 are positionable in the flexed position by the application of a biasing force on the flexible retainer members and the flexible retainer members 82 return to the at rest position when not subject to the biasing force. In FIG. 3, dashed outline 83 indicates a flexed position of a retainer member 82 when subjected to a biasing force as exemplified by arrow 81. It is noted that the biasing force indicated by arrow 81 is a radially outwardly directed force as might occur when removing tube 28 from space 79. Such a force may also be exerted by a user's finger when inserting tube 28 into space 79. Alternatively, the biasing force may push the retainer members 82 inwardly when inserting additional tubing into space 79. Pushing members 82 inwardly may also be used to remove tubing 28 and, in other embodiments, members 82 may be biased to the side to provide clearance for the entry and exit of tube 28.

Flexible tubing 28 wound about the central spindle 78 is retained within the toroidal space 79 by the plurality of flexible retainer members 82 when the retainer members are in the at rest position. Biasing the flexible retainer members 82 to the flexed position allows passage of the tubing 28 between the distal end of retainer members 82 and the base member 74 and thereby allows the length of the flexible tubing 28 wound about the central spindle 78 within the toroidal space to be increased or reduced. In assembly, the gap distance between the at-rest retainer members 82 and the base member 74 is less than a diameter of the tubing 28 to thereby block tubing 28 from exiting toroidal space 79 without application of the biasing force. In some embodiments, the retainer members 82 abut base member 74 in the at-rest position.

Alternatively, members 82 may be rigid members but have a shortened length that allows tube 28 to be slid into space 79 between the distal end of the member 82 and base member 74 without flexing the member 82. In this embodiment, it may also be advantageous that some (e.g., half) of the retainer members extend from upper member 76 and the remainder from base member 74 with the location of the retainer members 82 alternating to thereby facilitate the retention of tube 28 within space 79 by the retainer members 82. The use of such retainer members 82 not only facilitates retaining that portion of tube 28 wound about spindle 78 in toroidal space 79, but also facilitates retaining the angular position of tube 28 at the points where it enters and leaves toroidal space 79.

In the illustrated embodiment, retainer members 82 are all located on upper member 76 and extend toward base member 74. Alternative embodiments, however, may utilize retainer members that all extend from the base member toward the upper member, or, as mentioned above, have retainer members that extend from both the base member and upper member. Where all of the retainer members extend from only one of the upper member or base member, it may generally be advantageous for arcuate grooves 80 to be located on the opposite member as in the illustrated embodiment wherein all of the retainer members 82 extend from upper member 76 and grooves 80 are located on base member 74. This positioning of the retainer members 82 and grooves 80 positions the grooves near the unattached distal ends of retainer members 82 where passage of tube 28 will occur between the distal end of retainer members 82 and base member 74 when inserting or removing tube 28 from toroidal space 79 and thereby provide a convenient location for gripping tube 28.

In the illustrated embodiment, upper member 76 is not separable from the device. However, alternative embodiments employ an upper member that is detachable to thereby facilitate the extension or retraction of tube 28. In still other embodiments, spindle 78 may take the form of a telescoping element that allows upper member 76 to be axially moved relative to base member 74 without detachment of upper member 76 to thereby facilitate the extension or retraction of tube 28.

Flexibility in the length of the tube stored within toroidal space 79 is provided by the ability of the user to select how much of the tube to wrap about spindle 78 and to select which grooves 80 for securing the enter/exit of tube 28 from device 72. While FIG. 3 illustrates tube 28 extending less than one full wrap about spindle 78, the user may wrap tube 28 around spindle 78 several times to thereby manage a greater length of tube 28.

In the illustrative embodiment, upper member 76 and spindle 78 are rotationally fixed relative to base member 74 when coupled to base member 74. In an alternative embodiment, upper member 76 and spindle 78 may rotate relative to base member 74 as indicated by arrow 75. By providing a rotatable upper member 76 and spindle 78, the length of tube 28 disposed about the spindle 78 within the holding chamber may be adjusted by rotation of the upper member and spindle. A locking member may be actuated to hold the upper member 76 and spindle 78 in a static position relative to base member 74 to provide for the appropriate length of tubing 28.

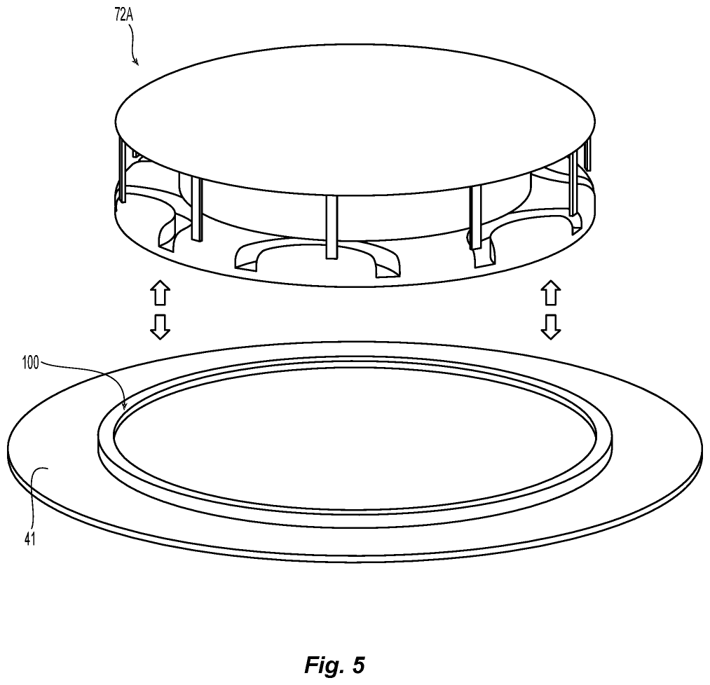

FIG. 5 illustrates a modified embodiment of the device of FIGS. 3 and 4. The device 72A illustrated in FIG. 5 has a design similar to that of device 72 but further includes a mounting member 100 to which the base member 74 is releasably attachable. In this embodiment, mounting member 100 is secured to the patient with an adhesive patch 41. In FIG. 5, device 72A is shown with the base member, central spindle and upper member assembled together and detached from mounting member 100. Device 72A may have its base member attached and detached from mounting member 100 while a length of tube 28 is still disposed within toroidal space 79.

The tube-holding portion (e.g., base member, central spindle, upper member, and retainer members) of the device 72A which is used to manage and secure a length of tubing 28 is detachable from mounting member 100. The ability to detach the tube-holding portion of the device 72A from mounting member 100 allows the user to disconnect tube 28 from the connection or insertion hub (FIGS. 1 and 2) and then remove the reservoir 22, associated delivery device, and tube 28 with the detached tube-holding portion without having to remove tube 28 from the device 72A. For example, the user may want to perform such a disengagement of the reservoir and pump when taking a bath. By being able to detach the tube-holding portion from the user's body without disengaging the tube 28 from the device 72A, the detachment and subsequent re-attachment process is made simpler and more convenient for the user.

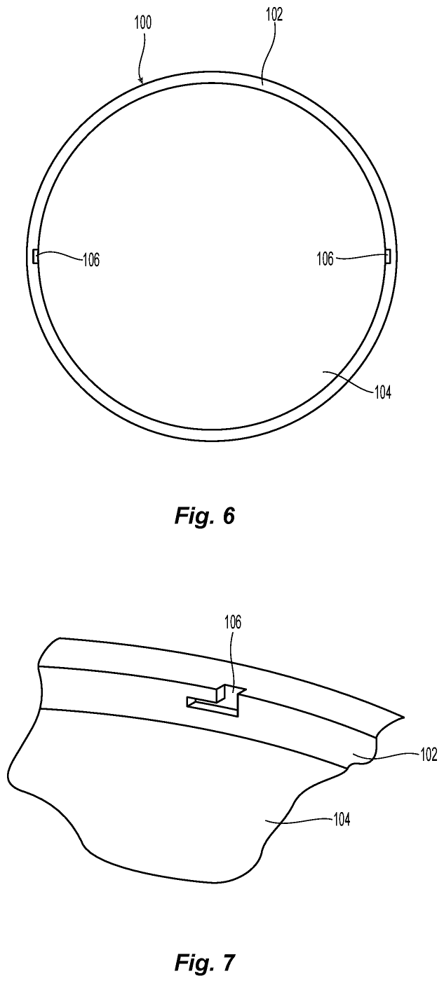

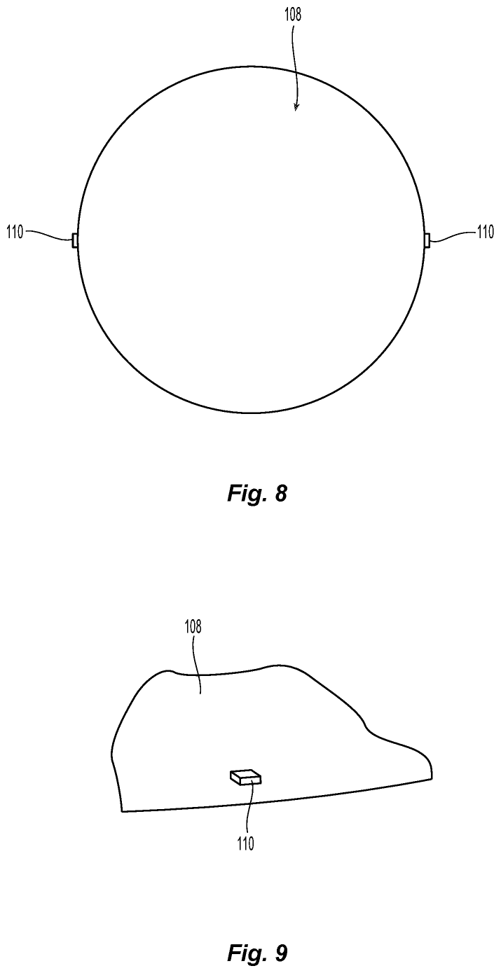

The releasable securement of the device 72A with the mounting member 100 may be accomplished using a number of different attachment techniques. FIGS. 6-9 illustrate one exemplary manner of releasably securing a holding portion 108 to a mounting member 100. In this embodiment, mounting member 100 includes a ring or collar 102 which extends upwardly from a bottom plate 104. Bottom plate 104 is readily adapted to have an adhesive layer (e.g., adhesive layer 41 of FIG. 5) disposed on its surface opposite ring 102. Ring 102 also includes two diametrically opposed L-shaped grooves 106 as illustrated in FIGS. 6 and 7. Holding portion 108 of FIGS. 8 and 9 includes two diametrically opposed tabs 110 that are inserted into grooves 106. Holding portion 108 is then rotated slightly to firmly engage tabs 110 into grooves 106 and releasably secure holding portion 108 to mounting structure 100. To remove, holding portion 108 is rotated in the opposite direction to a position that allows the withdrawal of tabs 110 from grooves 106.

For example, in the embodiment of FIG. 5, the base member, central spindle, upper member and plurality of retainer members form the holding portion 108 and tabs 110 may be located on the base member 74. This allows the mounting member 100 to be secured to the patient, for example, by adhesively securing it to the skin of the patient, while the base member, central spindle, upper member and plurality of retainer members, together with a length of flexible tubing, may be selectively secured and detached from the mounting member.

A variety of other attachment methods may also be employed. For example, cooperating helical threads, magnetic closures, an alternative bayonet type attachment, cooperating hook and loop patches, or other suitable attachment mechanisms may be employed.

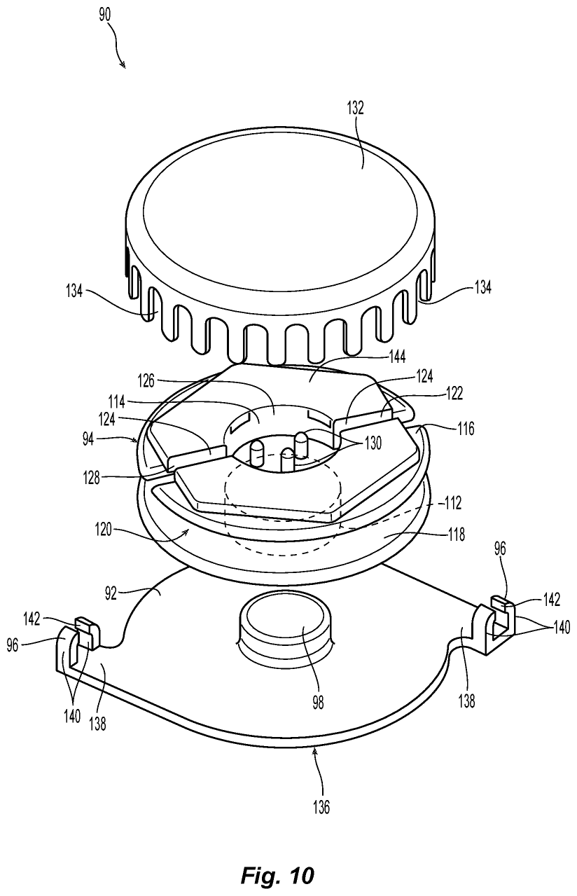

Another embodiment of a tube management device for an infusion set having a flexible tube for conveying medication between a reservoir and an infusion site on a patient is illustrated in FIGS. 10-13. Device 90 includes a support member 92 which can be secured to the patient. For example, the illustrated support member 92 has a layer of adhesive 136 on one side that can be used to adhesively secure support member 92 to the skin of the patient. Alternative attachment arrangements may also be used, for example, the device may have a clip that can be secured to the belt of the patient to thereby secure the device to the patient.

A reel member 94 is detachably mountable on support member 92 and provides a tube-holding portion for device 90. As further discussed below, a length of flexible tube 28 may be wound on reel member 94. Reel member 94 is rotatable relative to support member 92 when mounted on support member 92. Rotation of reel member 94 either retracts or extends the flexible tube 28 depending upon the direction in which reel member 94 is rotated. Reel member 94 is detachable from support member 92 with a section of flexible tubing 28 wound on reel member 94. As discussed herein, the ability to disengage a device from the patient while tubing 28 is engaged with the device is beneficial such as when the patient takes a bath or shower. Reel member 94 may comprise a single molded component or a plurality of components assembled together.

In the illustrated embodiment, support member 92 includes first and second tube guides 96 which loosely grip flexible tube 28 in a manner that allows tube 28 to slide through guides 96 as tube 28 is retracted or extended on reel member 94. As most easily seen in FIG. 10, support member 92 includes two ears 138 that extend outwardly with tube guides 96 disposed thereon. Tube guides 96 are located on opposite sides of reel member 94 when device 90 is fully assembled. Each tube guide 96 is formed by a pair of flexible arms 140 defining a slot 142 therebetween. Tube 28 may be forced through slot 142 to either engage or disengage tube 28 from guide 96. While the illustrated embodiment utilizes guides 96 that permit tube 28 to slide therethrough, alternative embodiments may employ guides that more tightly grip tube 28 in a manner that inhibits or prevents tube 28 from sliding relative to the guides. As can be seen in the figures, the illustrated tube guides 96 are positioned such that the tube 28 extending through the guides will slide in a direction through the guides along or parallel to a tangential line to the outer perimeter of toroidal space 120 within which tube 28 is wound about reel member 94.

Support member 92 also includes a central outwardly projecting shaft 98 on which the reel member 94 may be mounted. In the illustrated embodiment, reel member 94 includes a socket 112 that mates with shaft 98 to releasably secure reel member 94 on support member 92. Alternatively, support member 92 may define a socket for engaging a shaft on reel member 94, or another suitable means of rotatably mounting reel member 94 on support member 92 could be employed.

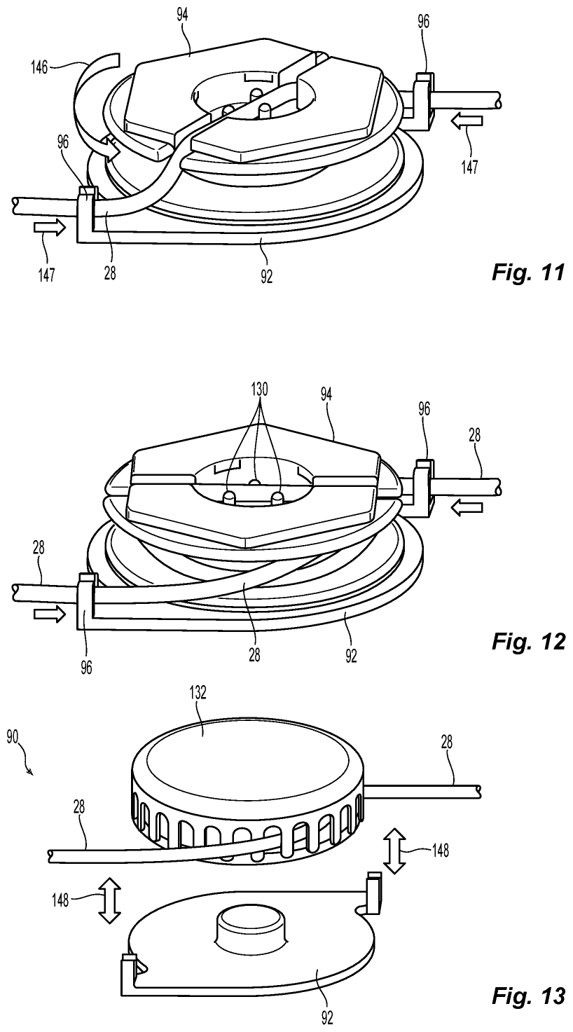

Reel member 94 includes a spindle section 114 and radially extending flanges 116, 118 which define a toroidal space 120 for receiving the flexible tube 28 as it is wound on the reel member 94. Flanges 116, 118 are illustratively parallel to each other. Reel member 94 also includes a central passage 122 extending through the spindle section 114 for receiving the flexible tube 28, as illustrated in FIGS. 11 and 12.

Central passage 122 defines an open side 124 that extends the full length of the central passage 122 thereby allowing the flexible tube 28 to be laid into the central passage at a non-terminal portion of the flexible tube. As those having ordinary skill in the art will recognize, when reel member 94 is rotated, the engagement of the tube 28 with central passage 122 causes this section of the tube to rotate with reel member 94 and thereby wind tube 28 about the spindle or dispense a previously wound section of tube 28 from the spindle.

In the illustrated embodiment, spindle section 114 also defines a hollow interior 126 with the central passage 122 being formed by diametrically opposite open slots 128 in reel member 94. Slots 128 are in communication with the hollow interior 126. A plurality of posts 130 are located in the hollow interior 126 such that, when the flexible tube is routed about the plurality of posts 130, tube 28 is securely gripped by posts 130 to thereby block the sliding movement of tube 28 relative to posts 130.

It is noted that neither socket 112 nor hollow interior 126 extends the full axial height of spindle section 114. For example, both portions may extend for slightly less than one half the axial height of spindle section 114 with a wall of material separating socket 112 from hollow interior 126 and supporting posts 130. One of the socket 112 and shaft 98 may include an annular recess with the other one of the socket 112 and shaft 98 including an annular projection that snap fits within the recess to retain the parts together. In this embodiment, the shaft 98 and socket 112 are formed out of a sufficiently flexible and resilient material to allow the two parts to be pulled apart when a moderate force is applied.

Reel member 94 includes non-circular section 144 located on the distal surface of top flange 116. Non-circular section 114 facilitates manual rotation of reel 94 when extending or retracting tube 28 on reel member 94. This section may also facilitate the attachment of a cap with the cap having a recessed area that mates with non-circular section 144. Non-circular section 144 is illustratively hexagonal in shape.

The illustrated embodiment includes a cap 132 that is detachably securable to the reel member 94. When cap 132 is secured to reel member 94, cap 132 surrounds toroidal space 79 that holds the length of tube 28 wrapped about reel member 94. Cap 132 defines a plurality of circumferentially spaced openings 134 which allow the passage of tube 28 through cap 132. Openings 134 have an open end whereby a non-terminal portion of tube 28 may be slid into openings 134 to allow passage of tube 28 through cap 132 during attachment of cap 132.

FIG. 13 illustrates cap 132 mounted on reel member 94 with a length of tube 28 wound on reel member 94. Cap 132 retains the length of tube 28 wound on reel member 94 in toroidal space 120 when cap 132 is attached to reel member 94. Tube 28 extends through openings 134 at the two points where tube 28 enters/exits reel member 94. Cap 132 may remain attached to reel member 94 when reel member 94 is secured to support member 92 and when reel member 94 is detached from support member 92. Arrows 148 in FIG. 13 indicate that reel member 94 may be attached and detached from support member 92 with a tube 28 wound thereon and a cap 132 secured to reel member 94. The ability to leave cap 132 on reel member 94 when attaching and detaching reel member 94 from support member 92 provides convenience to the user when removing tube 28 and reel member 94 from their body. Cap 132 may also be removed from reel member 94 when reel member 94 is either attached or detached from support member 92.

The operation of use of device is best understood with reference to FIGS. 10-13. When first engaging tube 28 with device 90, cap 132 is removed and tube 28 is engaged by laying it into central passage 122 through open side 124 and positioning it between posts 130. Tubing 28 is also engaged with each of the tube guides 96 on opposite sides of central passage 122. In this regard, it is noted that tube 28 does not have to be threaded through any part of device 90, and a non-terminal portion of tube 28 may be operably engaged with and disengaged from device 90 while tube 28 is operably connected to convey medication between the reservoir and the infusion site.

To wind an excess of tubing onto device 90, reel member 94 is manually rotated as indicated by arrow 146 in FIG. 11. In this regard, it is noted that tube guides 96 are arranged for reel member 94 to be rotated in the counter-clockwise direction as viewed from the top of device 90 to wind additional tubing onto reel member 94. In alternative embodiments, however, tube guides 96 may be arranged to rotate the reel member in the opposite direction. In still other embodiments, tube guides 96 may be omitted or be rotatable on the support member whereby reel member 94 may be rotated in either direction when starting to wind up tube 28 thereon.

In the embodiment of FIG. 11, as reel member 94 is rotated in direction 146 to wind tube 28 onto the reel member 94, tube 28 slides through each of tube guides 96 as it is wound on the reel member as indicated by arrows 147. Once the desired length of tube 28 has been wound on reel member 94, cap 132 is attached to reel member 94.

To remove some or all of the tube 28 from the reel member 94, the cap 132 is removed and reel member 94 is rotated in the direction opposite that indicated by arrow 146 to thereby allow tubing to slide outwardly away from reel member 94 through tube guides 96. Alternatively, the user may simply grab tube 28 at locations outside tube guides 96 and pull the tubing outwardly which will cause reel member 94 to rotate as tube 28 is extended from the reel member. In some embodiments, it may be possible to pull tube 28 outwardly in this manner while cap 132 is still attached to reel member 94. Reel member 94 may include a detent system or other mechanism to resist rotating in the unwinding direction unless an outward force applied by the user exceeds a threshold.

It is further noted that support member 92 may be secured to the patient when engaging tube 28 with device 90, or support member 92 may be secured to the patient after winding a length of tube 28 onto device 90.

In one embodiment, a torsional spring may be used to bias reel member 94 relative to support member 92. Such a spring-biased assembly may be employed to automatically wind up excess tube length. A releasable locking member may be employed to hold the reel member in a static position relative to support member 92 to provide for the appropriate length of tubing 28.

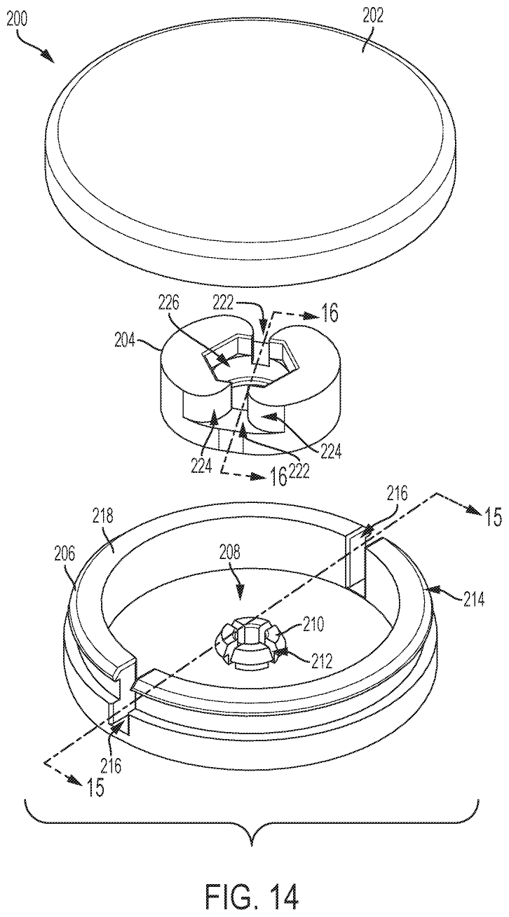

Another embodiment of a tube management device 36 (FIG. 1) for an infusion set having a flexible tube for conveying medication between a reservoir and an infusion site on a patient is illustrated in FIGS. 14-18. Tube management device 200 includes an upper member 202, a base member 206, and a central spindle 204 positioned between base member 206 and upper member 202. In the illustrated embodiment, upper member 202 and spindle 204 are rotatably mounted on base member 206.

Base member 206 includes a centrally-positioned post or shaft 208 with a radial post-rim 210 that protrudes radially outwardly beyond the main diameter of post 208. Post 208 illustratively includes one or more post-cutouts 212 that cut transversely through post 208, thereby separating post 208 into several (e.g., four) portions each fixed at the bottom to base 206 and that may be flexed radially inward to receive spindle 204. Base 206 also has a tubing slot 216 cutting transversely through its radial wall 218 for receiving tubing through diametrically opposed portions of wall 218. Radial wall 218 includes a wall rim 214 extending circumferentially around base member 206 and radially outwardly from wall 218.

Illustratively, central spindle 204 has a generally round or oval outer profile and includes a tubing slot 222 that extends transversely through the center of spindle 204, providing two diametrically opposed slots 222 for receiving tubing. Walls forming the tubing slots 222 illustratively include fillets 224 at either end having radius R, where R is larger than the minimum bend-radius of the tubing that is inserted into the slots 222. Spindle 204 also has a central opening 226 extending through the body of spindle 204. An upper portion of opening 226 serves as a keyhole (illustratively hexagonally shaped) configured to mate with a corresponding feature 236 (FIG. 18) formed on the bottom surface of upper member 202, as described herein. A lower portion of opening 226 is sized to receive post 208 of base member 206. Spindle 204 may optionally include a gutter, illustratively a circumferential gutter 228 in FIG. 16, which is recessed in the bottom surface of spindle 204.

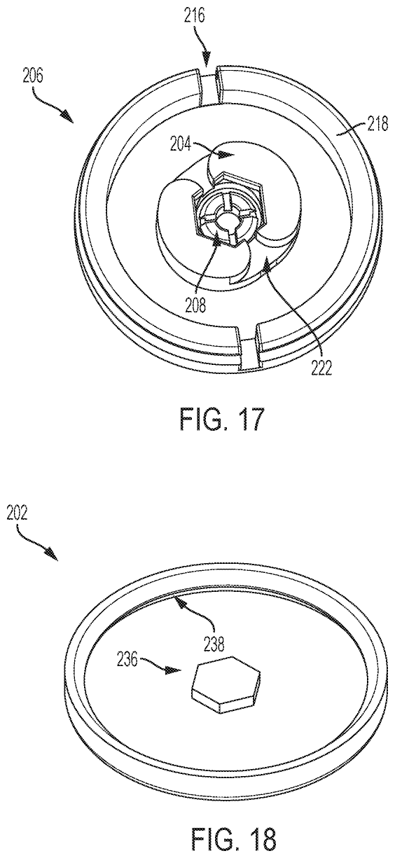

During manufacturing, spindle 204 is assembled to base member 206 by pushing an interference chamfer 230 (FIG. 16) of spindle 204 down over post rim 210 onto post 208 of base member 206. An assembled spindle 204 and base member 206 is illustrated in FIG. 17. The post-cutouts 212 (FIG. 14) allow post 208 to flex radially inward, thereby securing spindle 204 to base member 206 (and blocking removal) via the interaction of interference chamfer 230 and post rim 210.

Referring to FIG. 18, upper member 202 includes keyed feature 236 that mates to the corresponding keyed feature (i.e., lid-keyhole 226) in spindle 204. Upper member 202 also includes a circumferential rim 238 that mates to the wall rim 214 on base member 206. In one embodiment, at least one of rim 238 and wall rim 214 are adapted to flex radially to facilitate assembly of upper member 202 to base member 206.

In an exemplary method of operation with an infusion set tubing, with upper member 202 removed the user first rotates spindle 204 relative to base member 206 to align tubing slots 216, 222. The user then inserts the infusion set tube through the aligned tubing slots 216, 222. Upper member 202 is then attached to base member 206 by mating rim 238 to wall rim 214, thereby securing the lid to the base. Feature 236 is aligned with spindle 204 to mate with the top portion of opening 226 of spindle 24, thereby mechanically coupling spindle 204 to upper member 202. In the exemplary embodiment, upper member 202 is free to rotate relative to base 206, and by rotating upper member 202, spindle 204 is also rotated causing the tubing to be wound around the spindle 204. To increase the tube length outside device 200, the user pulls on one or both ends of the tube.

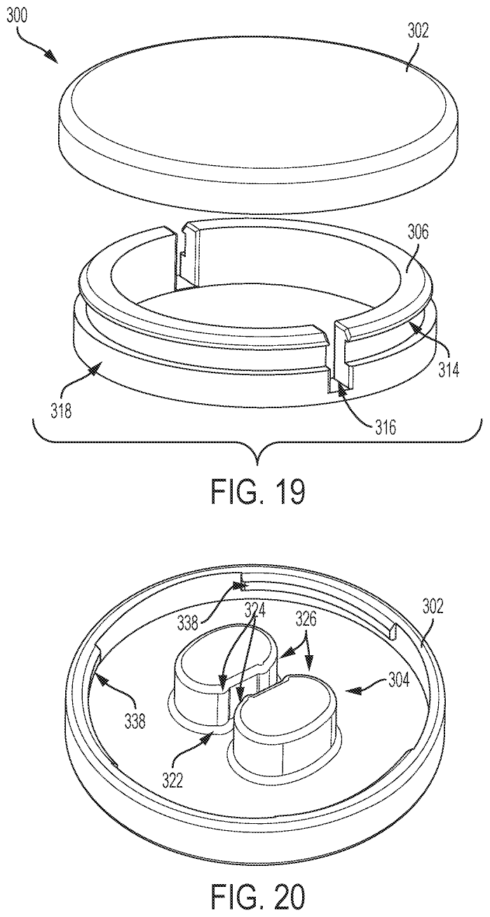

Another embodiment of a tube management device 36 (FIG. 1) for an infusion set having a flexible tube for conveying medication between a reservoir and an infusion site on a patient is illustrated in FIGS. 19 and 20. Tube management device 300 includes an upper member 302, a base member 306, and a central spindle 304 (FIG. 20) integrated with the upper member 302 on the lower or inside surface of upper member 302. In the illustrated embodiment, upper member 302 and spindle 304 are rotatably mounted on base member 306.

Base member 306 is similar to base 206 of FIG. 14 but optionally does not include the post 208 of base 206. In another embodiment, base 306 does include a centrally located post configured to couple to spindle 304. As illustrated, base 306 has a tubing slot 316 cutting transversely through its radial wall 318 for receiving tubing through diametrically opposed portions of wall 318. Radial wall 318 includes an outer wall rim 314 extending circumferentially around base member 306 and radially outwardly from wall 318.

Referring to FIG. 20, upper member 302 includes an interior circumferential rim 338 that mates to the wall rim 314 on base member 306. Rim 338 may encircle the full circumference of upper member 302 or, as illustrated in FIG. 20, comprise multiple portions each having an arc length. In one embodiment, at least one of rim 338 and wall rim 314 are adapted to flex radially to facilitate assembly of upper member 302 to base member 306.

Central spindle 304 illustratively includes a pair of lobes or posts integrated with and protruding from the bottom surface of upper member 302. The lobe pair forms a central tubing slot 322 that extends transversely through the center of spindle 304 for receiving tubing. Tubing slot 322 includes tubing retention features 324 formed in the walls of spindle 304. The walls forming the tubing slot 322 also illustratively include fillets 326 at either end having radius R, where R is larger than the minimum bend-radius of the tubing that is inserted into the slots 322.

In an exemplary method of operation with an infusion set tubing, with upper member 302 removed the user first inserts the infusion set tube through the tubing slot 322 of spindle 304. Upper member 302 is then attached to base member 306 by mating rim 338 to wall rim 314 while aligning slots 316 and slot 322, thereby allowing tubing to exit device 300 through slots 316 and securing the lid 302 to the base 306. In the exemplary embodiment, upper member 302 is free to rotate relative to base 306, and by rotating upper member 302, spindle 304 is also rotated causing the tubing to be wound around the spindle 304. To increase the tube length outside device 300, the user pulls on one or both ends of the tube.

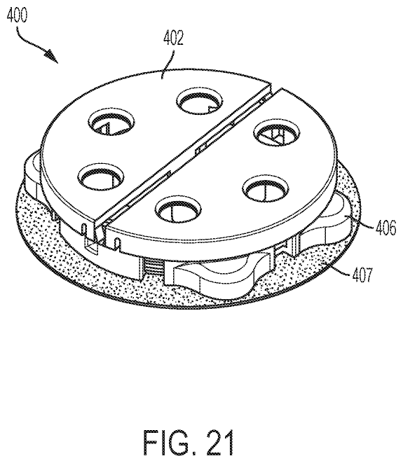

Another embodiment of a tube management device 36 (FIG. 1) for an infusion set having a flexible tube for conveying medication between a reservoir and an infusion site on a patient is illustrated in FIGS. 21-23. Tube management device 400 includes an upper member 402, a base member 406, and a central spindle 404 positioned between members 402, 406. Base member 406 includes an illustratively circular-shaped adhesive member or patch 407 for securing device 400 to a patient's skin.

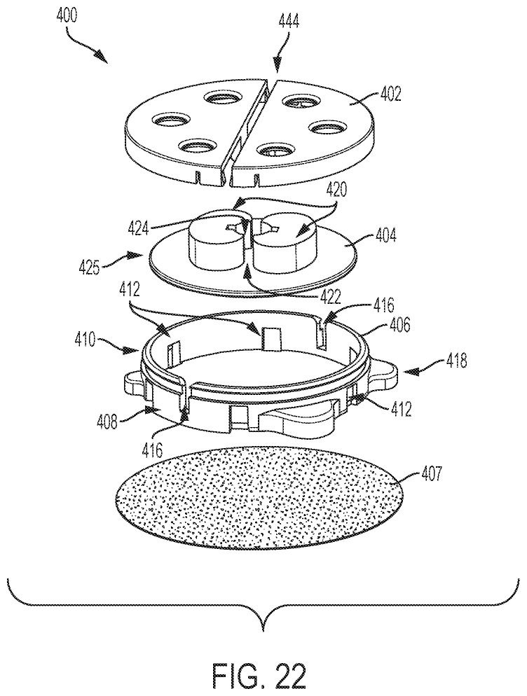

Referring to FIG. 22, base member 406 includes a radial wall 408 and a radial rim 410. Base 406 includes a pair of diametrically opposed tubing slots 416 each with a chamfered top end. Base 406 also includes a plurality of drain slots 412 circumferentially spaced around radial wall 408. For example, these slots 412 facilitate draining such that if a user wears the device in water (e.g. when bathing or swimming), the water has a free path to drain out of the device. Base 406 also has optional radial protrusions 418 serving as finger holds which help a user hold base 406 while lid 402 is rotated relative to base 406. Finger holds 418 and drain slots 412 may also be provided on devices 200, 300 described herein.

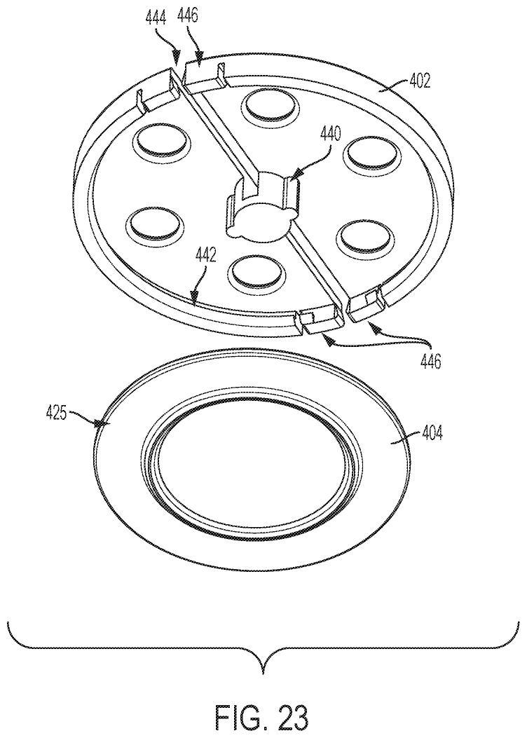

Spindle 404 has an outer profile that is generally round or oval in shape. Spindle 404 includes a pair of posts 420 divided by a tube slot 422 which may optionally have tubing retention features formed in the walls of the posts 420, as similarly described herein with respect to features 324 of device 300 in FIG. 20. As described with spindle 304 of device 300, the walls forming tube slot 422 of spindle 404 have fillets at either end having radius R, where R is larger than the minimum bend-radius of the tubing that will be inserted into the slot 422. In the illustrated embodiment, spindle 404 also includes a bottom circular flange 425 which is smaller (illustratively only slightly smaller) than the inner diameter of base 406 to allow flange 425 to fit within base 406. Spindle further includes a key-slot opening 424 formed in the posts 420 for mating to a corresponding key feature 440 (FIG. 23) in lid 402.

Upper member 402 has a circumferential lid-rim 442 which may encircle the entire circumference of member 402 (as shown in FIG. 23) or may be broken into segments such that each segment extends for only a portion having an arc length. Upper member 402 also contains a tubing slot 444 and tubing gates 446 which allow a tube to be inserted one way into tubing slot 444 but block or restrict the tube from coming back out of slot 444 without extra force by the user to compress the tube through gates 446. Tubing slot 444 illustratively extends across the diameter of member 402 with a tubing gate 446 at each end of the slot to retain the tubing in the slot 444.

During manufacturing, the keying feature 440 on member 402 mates with the corresponding keying feature 424 (e.g., keyhole features) on spindle 404 to form a lid/spindle sub-assembly. The lid/spindle sub-assembly is then attached to base member 406 via engagement of rims 410 and 442.

In an exemplary method of operation with an infusion set tubing, the upper member 402, base member 406, and spindle 404 are pre-assembled. As such, the user is only required to first align tubing slot 422, 444 in the lid/spindle sub-assembly with tubing slots 416 in base 406 prior to inserting the tubing. The user then inserts the tube through tubing slot 444 in lid 402 and down into slots 416 in base 406. Tubing gates 446 in lid 402 allow the tube to be pushed through (e.g., compressed) into slot 444, but tubing gates 446 block or restrict the tube from coming back up out of slots 444 and slots 416 in base 406, as described herein. Lid 402 is free to rotate with respect to base 406, and by turning lid 402, the user also turns spindle 404, causing the tubing wind around spindle 404. To lengthen the tube outside of device 400, the user pulls on one or both ends of the tube.

The embodiments disclosed herein may have an optional adhesive patch that allows the device to be worn on body. When worn on-body, the devices of the present disclosure also provide strain relief for the insertion site; if the infusion set tube is pulled on the pump side of the device, the strain will be communicated to the tubing management device rather than to the insertion site.

In some embodiments, the user may attach the device to his or her body in a reversible manner, so that the entire infusion set may be removed and re-attached without unwinding the tube from the tubing-management device. Such a scheme may be realized in several ways. In one example, a thin hook-and-loop (e.g., Velcro) interface is positioned between the device and an adhesive patch worn on-body. In another example, the device mates with a clip or clasp that is worn on-body, such as illustrated in FIGS. 10-13. Other means of securing the device to provide strain relief may also be provided, such as using a belt-clip.

The infusion sets described herein may further comprise a drug. In another embodiment, a system may comprise one or more devices including the infusion set and a drug. The term “drug” refers to one or more therapeutic agents including but not limited to insulins, insulin analogs such as insulin lispro or insulin glargine, insulin derivatives, GLP-1 receptor agonists such as dulaglutide or liraglutide, glucagon, glucagon analogs, glucagon derivatives, gastric inhibitory polypeptide (GIP), GIP analogs, GIP derivatives, oxyntomodulin analogs, oxyntomodulin derivatives, therapeutic antibodies and any therapeutic agent that is capable of transport or delivery by the infusion set. The drug as used in the infusion set may be formulated with one or more excipients. The infusion set is operated in a manner generally as described above by a patient, caregiver or healthcare professional to deliver drug to a person.

While this invention has been described as having exemplary designs, the present invention may be further modified within the spirit and scope of this disclosure. This application is therefore intended to cover any variations, uses, or adaptations of the invention using its general principles.

What is claimed is:

1. A tube management device for an infusion set having a flexible tube for conveying a medication between a reservoir and an infusion site on a patient, the device comprising:
a central spindle defining an axis;
a base member projecting radially outwardly from the central spindle;
an upper member projecting radially outwardly from the central spindle wherein the base member and the upper member are axially spaced apart;
a plurality of retainer members disposed on at least one of the base member and the upper member, wherein the central spindle, the base member, the upper member, and the plurality of retainer members define a toroidal space sized to receive a variable length of the flexible tube; and
wherein the plurality of retainer members restrict radially outward movement of a length of the flexible tube wound about the central spindle.

2. The tube management device of claim 1, wherein at least one of the base member and the upper member defines a plurality of arcuate grooves adapted to grippingly receive the flexible tube.

3. The tube management device of claim 1, wherein the plurality of retainer members are resiliently flexible between an at rest position and a flexed position, the plurality of retainer members being positionable in the flexed position by the application of a biasing force on the retainer members and the plurality of retainer members being configured to return to the at rest position from the flexed position when not subject to the biasing force, and wherein biasing the retainer members to the flexed position allows the length of the flexible tube wound about the central spindle within the toroidal space to be increased or reduced.

4. The tube management device of claim 1, wherein the plurality of retainer members extend from the upper member towards the base member and are circumferentially spaced on the upper member, and the base member defines a plurality of arcuate grooves adapted to grippingly receive the flexible tube.

5. The tube management device of claim 1, further comprising a mounting member releasably securable to the base member and configured to attach to the patient, wherein the base member, the central spindle, the upper member, the plurality of retainer members, and the length of the flexible tube wound about the central spindle form a removable assembly adapted to be selectively attached to and detached from the mounting member.

6. The tube management device of claim 5, wherein the mounting member includes an adhesive layer for securing the mounting member to the skin of the patient.

7. A tube management device for an infusion set having a flexible tube for conveying medication between a reservoir and an infusion site on a patient, the device comprising:
a support member adapted to be secured to the patient;
a reel member rotatably mountable on the support member and adapted to receive the flexible tube, wherein, when the reel member is mounted on the support member, a rotation of the reel member in a first direction is operable to wind the flexible tube onto the device and a rotation of the reel member in a second direction is operable to extend the flexible tube from the device, and wherein the reel member is detachable from the support member with a length of flexible tube wound on the reel member.

8. The tube management device of claim 7, wherein the support member is attachable to the patient and wherein a non-terminal portion of the flexible tube is configured to be operably engaged with and disengaged from the device when the flexible tube is operably connected to convey medication between the reservoir and the infusion site.

9. The tube management device of claim 8, wherein the support member is adhesively securable to the skin of the patient.

10. The tube management device of claim 7, further comprising first and second tube guides configured to slidably grip the flexible tube wherein the flexible tube is slideable through the first and second tube guides when being retracted and extended on the reel member.

11. The tube management device of claim 10, wherein the first and second tube guides are positioned on opposite sides of the reel member such that the tube slides in each of the first and second tube guides in a direction tangentially to a toroidal space defined by the reel member.

12. The tube management device of claim 7, wherein the support member includes an outwardly projecting shaft, the reel member being rotatably mountable on the shaft.

13. The tube management device of claim 7, wherein the reel member includes a spindle section and first and second radially extending flanges which define a toroidal space for receiving the flexible tube as it is wound on the reel member and wherein the reel member further defines a central passage extending through the spindle section for receiving the flexible tube.

14. The tube management device of claim 13, wherein the central passage defines an open side extending a full length of the central passage to allow the flexible tube to be inserted into the central passage at a non-terminal portion of the flexible tube.

15. The tube management device of claim 14, wherein the spindle section defines a hollow interior and the central passage is formed by diametrically opposed open slots formed in the reel member in communication with the hollow interior.

16. The tube management device of claim 15, wherein the hollow interior of the spindle section includes a plurality of posts wherein, when the flexible tube is routed about the plurality of posts, the flexible tube is gripped by the plurality of posts to block sliding movement of the flexible tube relative to the plurality of posts.

17. The tube management device of claim 7, further comprising a cap attachable to the reel member, wherein when the cap is secured to the reel member the cap surrounds a toroidal space for receiving the flexible tube as the flexible tube is wound on the reel member, and wherein the cap defines a plurality of circumferentially spaced openings, each of the openings being adapted to allow passage of the tube through the cap.

18. The tube management system of claim 17, wherein the cap is attachable to the reel member when the reel member is mounted on the support member and when the reel member is detached from the reel member, and the cap facilitates retention of the flexible tube that is wound on the reel member and disposed within the toroidal space when the cap is secured to the reel member.

19. The tube management device of claim 7, wherein the support member is adhesively securable to the skin of the patient and comprises first and second tube guides, the first and second tube guides slidably gripping the flexible tube such that the flexible tube is slideable through the first and second tube guides when being retracted and extended on the reel member; and
wherein the reel member includes a spindle section and first and second radially extending flanges which define a toroidal space for receiving the flexible tube as the flexible tube is wound on the reel member, and wherein the reel member further defines a central passage extending through the spindle section for receiving the flexible tube.
